(19) United States
(12) Patent Application Publication
(10) Pub. No.: US 2021/0267618 A1
(43) Pub. Date: Sep. 2, 2021

(54) MEDICAL TREATMENT TOOL

(71) Applicant: OLYMPUS CORPORATION, Tokyo (JP)

(72) Inventor: Noriaki YAMANAKA, Tokyo (JP)

(73) Assignee: OLYMPUS CORPORATION, Tokyo (JP)

(21) Appl. No.: 17/325,387

(22) Filed: May 20, 2021

Related U.S. Application Data
(63) Continuation of application No. PCT/JP2018/044148, filed on Nov. 30, 2018.

(51) Int. Cl. A61B 17/29

(52) U.S. Cl. CPC A61B 17/29; A61B 2017/2913; A61B 2017/2905; A61B 2017/2904

(57) ABSTRACT

A medical treatment tool includes: a forceps; an operating section; a sheath; first and second sections bending the sheath; and at least two wires passing through the first and second sections and transmitting, to the forceps, a force applied to the operating section. The sheath includes lumens allowing the wires to pass therethrough. With respect to a position of lumens at which the first and second sections are bent in opposite directions in the same plane, the lumens are twisted between the first and second sections in the same direction by the same angle that operating direction of the operating section is twisted relative to the opposite direction mentioned above. The first and second sections are bent in directions in which changes in path lengths of the wires by bending of the first bending section are offset by changes in the path lengths by bending of the second bending section.

### CROSS-REFERENCE TO RELATED APPLICATIONS

This is a continuation of International Application PCT/JP2018/044148 which is hereby incorporated by reference herein in its entirety.

### TECHNICAL FIELD

The present invention relates to a medical treatment tool.

### BACKGROUND ART

There is a well-known rigid manipulator in which paths of a plurality of wires that pass through a joint arranged at a distal end of a rigid insertion section to drive a movable section, such as forceps, arranged at a more distal side than the joint are twisted at the position of the joint, whereby the difference in path-length change between the paths due to bending of the joint is compensated for (for example, refer to PTL 1).

In order to compensate for the difference in path-length change between the paths due to bending of the joint by twisting the paths of the wires at the joint, it is necessary to twist the paths at an extremely short pitch within the length of the joint, resulting in an increase in friction between the wires and their paths and accordingly making it difficult to transmit a force by means of the wires.

### CITATION LIST

### Patent Literature

### {PTL 1}

Japanese Translation of PCT International Application, Publication No. 2013-5185665

### SUMMARY OF INVENTION

One aspect of the present invention is a medical treatment tool including: a forceps that treats an affected area; an operating section including a handle and a dial, and configured to operate the forceps; a guide sheath arranged between the forceps and the operating section; a first bending section and a second bending section arranged between the operating section and the forceps in a manner spaced apart from each other in a longitudinal direction, the first bending section and the second bending section bending the guide sheath; at least two treatment tool wires that pass through the first bending section and the second bending section of the guide sheath and that transmit, to the forceps, a force applied to the operating section; and at least two bending wires that pass through the first bending section and the second bending section of the guide sheath and that are fixed to the operating section and the forceps, the bending wires causing the guide sheath to be bent by operating the operating section, wherein the guide sheath includes a multi-lumen tube with a plurality of lumens that allow the treatment tool wires to pass therethrough, with respect to a position of lumens at which the first bending section and the second bending section are bent in opposite directions on the same plane, the lumens of the guide sheath are twisted between the first bending section and the second bending section in the same direction by the same angle that operating direction of the operating section is twisted relative to the opposite direction mentioned above, and the first bending section and the second bending section are bent in directions in which changes in path lengths of the treatment tool wires in the guide sheath caused by bending of the first bending section are offset by changes in the path lengths of the treatment tool wires in the guide sheath caused by bending of the second bending section.

Another aspect of the present invention is a medical treatment tool including: a forceps that treats an affected area; an operating section including a handle and a dial, and configured to operate the forceps; a guide sheath arranged between the forceps and the operating section; a first bending section and a second bending section arranged between the operating section and the forceps in a manner spaced apart from each other in a longitudinal direction, the first bending section and the second bending section bending the guide sheath; at least two treatment tool wires that pass through the first bending section and the second bending section of the guide sheath and that transmit, to the forceps, a force applied to the operating section; a bending sheath that allows the guide sheath to pass therethrough; and at least two bending wires that pass through the bending sheath and that are fixed to a distal end and a proximal end of bending sheath, the bending wires causing the guide sheath to be bent by operating the operating section, wherein the bending sheath is bent by bending the second bending section by operating the operating section, and the first bending section is bent according to bending of the bending sheath, the guide sheath includes a multi-lumen tube with a plurality of lumens that allow the treatment tool wires to pass therethrough, the forceps and the guide sheath are arranged so as to be movable back and forth in the longitudinal direction relative to the bending sheath, twist of the lumens of the guide sheath is arranged to be kept between the first bending section and the second bending section, and the first bending section and the second bending section are bent in directions in which changes in path lengths of the treatment tool wires in the guide sheath caused by bending of the first bending section are offset by changes in the path lengths of the treatment tool wires in the guide sheath caused by bending of the first bending section.

### BRIEF DESCRIPTION OF DRAWINGS

FIG. 1 is a schematic diagram showing a medical treatment tool according to one embodiment of the present invention.

FIG. 2 is a schematic diagram showing a state in which a distal bending section is bent by bending an operating bending section of the medical treatment tool in FIG. 1.

FIG. 3 is a transverse sectional view showing a guide sheath of the medical treatment tool in FIG. 1.

FIG. 4 is a longitudinal sectional view showing a first modification of the medical treatment tool in FIG. 1.

FIG. 5 is a transverse sectional view of the guide sheath and a bending sheath of the medical treatment tool in FIG. 4.

FIG. 6 is a schematic diagram showing a second modification of the medical treatment tool in FIG. 1.

FIG. 7 is a longitudinal sectional view showing a modification of the guide sheath in the medical treatment tool in FIG. 1.

FIG. 8 is a longitudinal sectional view showing a modification of the guide sheath in the medical treatment tool in FIG. 1.

FIG. 9 is a schematic diagram showing a third modification of the medical treatment tool in FIG. 1.

FIG. 10 is a schematic transverse sectional view of the guide sheath, illustrating a path-length change in the medical treatment tool in FIG. 1.

FIG. 11 is a schematic longitudinal sectional view of the guide sheath in FIG. 10.

FIG. 12 is a schematic diagram showing a fourth modification of the medical treatment tool in FIG. 1.

FIG. 13 is a schematic diagram showing a fifth modification of the medical treatment tool in FIG. 1.

### DESCRIPTION OF EMBODIMENTS

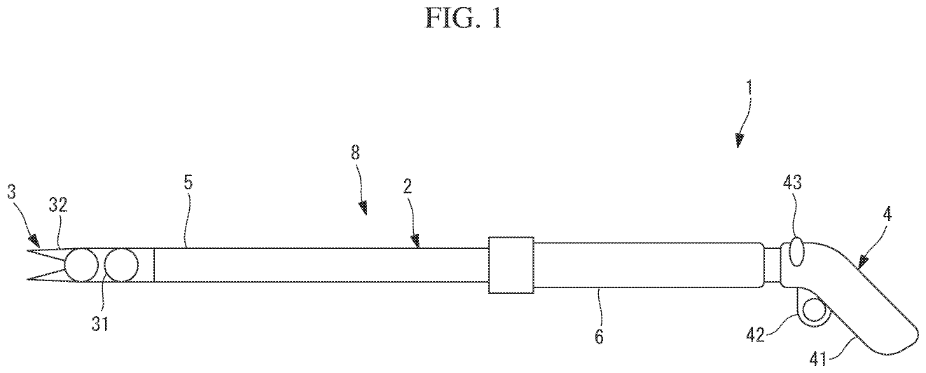

As shown in FIG. 1, a medical treatment tool 1 according to one embodiment of the present invention includes a shaft 2 extending in a straight line, a treatment section 3 arranged at a distal end of the shaft 2, and an operating section 4 that is arranged at a proximal end of the shaft 2 and that is used to operate the treatment section 3.

Between the treatment section 3 and the shaft 2 is a distal bending section (first bending section) 5 that swivels in two-dimensional directions to change the orientation of the treatment section 3 relative to the shaft 2. In addition, between the operating section 4 and the shaft 2 is an operating bending section (second bending section) 6 for supporting the operating section 4 so that the operating section 4 is swivelable in two-dimensional directions.

The treatment section 3 is, for example, grasping forceps and includes a joint 31 arranged at a more distal position than the distal bending section 5 and a grasping section 32 arranged at the distal end side of the joint 31.

The operating section 4 includes a handle 41 gripped by an operator, as well as a trigger 42 and an operating knob 43 that are provided on the handle 41.

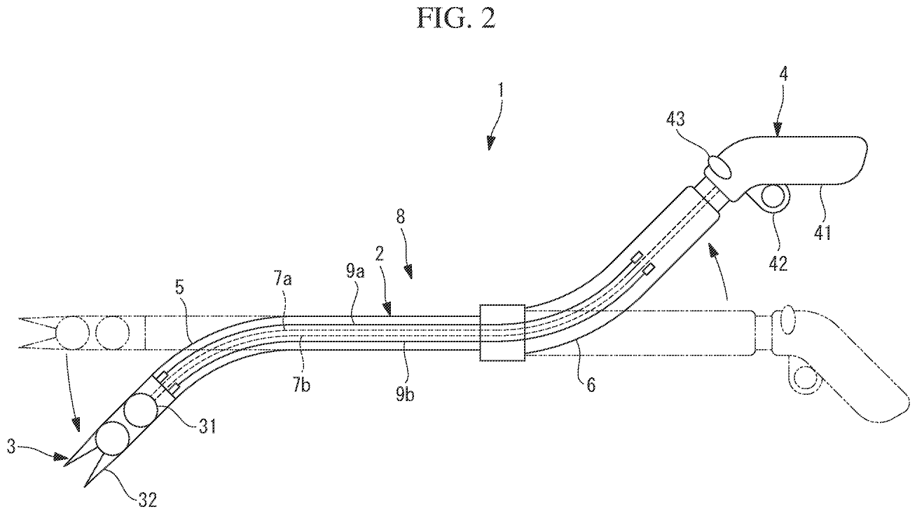

The operating knob 43 is connected to the joint 31 via treatment tool wires (force transmission members) 7a indicated by a broken line in FIG. 2 and can make the joint 31 of the treatment section 3 swivel by means of a tensile force that acts on the treatment tool wires 7a according to an operation of the operating knob 43. In addition, the trigger 42 is connected to the grasping section 32 via other treatment tool wires (force transmission members) 7b and can open and close the grasping section 32 by means of a tensile force that acts on the treatment tool wires 7b according to an operation of the trigger 42.

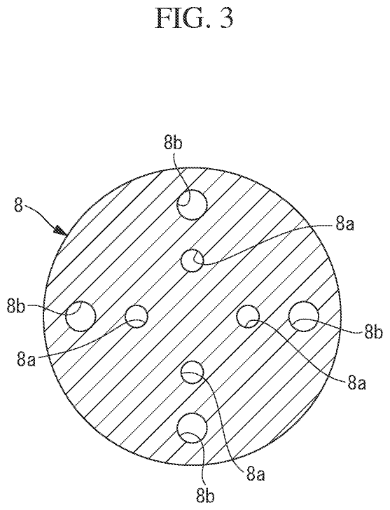

The handle 41, the operating bending section 6, the shaft 2, and the distal bending section 5, which extend from the operating section 4 to the treatment section 3, are formed of a guide sheath 8 in the form of a multi-lumen tube with a plurality of lumens 8a and 8b, as shown in FIG. 3. In the example shown in FIG. 3, the lumens 8a that allow the treatment tool wires 7a and 7b to pass therethrough are arranged at four radially inward locations, and the lumens 8b that allow bending wires 9a and 9b for connecting the operating section 4 to the treatment section 3 to pass therethrough are provided at four radially outward locations.

The operating section 4 and the shaft 2 are formed of a rigid guide sheath, and the operating bending section 6 and the distal bending section 5 are formed of a soft guide sheath having flexibility. The rigid guide sheath and the soft guide sheath may be formed seamlessly and integrally or may be formed by being bonded to each other with an adhesive or the like.

As shown in FIG. 3, the lumens 8b that allow the bending wires 9a and 9b to pass therethrough and the lumens 8a that allow the treatment tool wires 7a and 7b to pass therethrough are arranged at four equally-spaced locations in respective circumferential directions about the center of the guide sheath 8. As shown in FIG. 2, one end of each of the bending wires 9a and 9b is fixed to the operating section 4, and the other end of each of the bending wires 9a and 9b is fixed to the treatment section 3. The lumens 8a and 8b that allow the bending wires 9a and 9b and the treatment tool wires 7a and 7b to pass therethrough are arranged at upper/lower locations and at left/right locations of the shaft 2 and connect the operating section 4 to the treatment section 3 along the longitudinal direction of the guide sheath 8 with the phases thereof being maintained.

The operation of the medical treatment tool 1 according to this embodiment with the above-described structure is described below.

When an affected area is to be treated by using the medical treatment tool 1 according to this embodiment, the medical treatment tool 1 is inserted into the body from the treatment section 3 side under endoscopic observation, and the treatment section 3 is then made to face the affected area.

Thereafter, the operator grips the handle 41 arranged outside the body and swivels the operating section 4 in a desired direction relative to the shaft 2 so as to bend the operating bending section 6.

Because the end sections of the four bending wires 9a and 9b are fixed to the operating section 4, the tensile forces of the bending wires 9a and 9b located outside the curve formed by the bending of the operating bending section 6 increase, and the tensile forces of the bending wires 9a and 9b located inside the curve formed by the bending of the operating bending section 6 decrease.

In this embodiment, the bending wires 9a and 9b are made to pass through the lumens 8b provided in the guide sheath 8, and both ends of each of the bending wires 9a and 9b are fixed to the handle 41 and the treatment section 3, respectively. Hence, the bending wires 9a and 9b having increased tensile forces pull the treatment section 3 and bend the distal bending section 5.

Because the lumens 8b in the guide sheath 8 are arranged between the operating section 4 and the treatment section 3 with the phases thereof with respect to the center of the shaft 2 remaining the same, the bending wires 9a and 9b that are arranged outside the curve formed by the bending at the operating bending section 6 are arranged inside the curve formed by the bending at the distal bending section 5. As a result, as shown in FIG. 2, tilting the operating section 4 by an angle in one direction causes the distal bending section 5 to be bent by the same angle in the opposite direction in the same bending plane (plane orthogonal to the bending axis) as that of the operating bending section 6.

Thereafter, the operator operates the operating knob 43 and the trigger 42 provided on the handle 41 to manipulate the joint 31 and the grasping section 32 provided on the treatment section 3, thus treating the affected area.

In this case, at each of the operating bending section 6 and the distal bending section 5, the path lengths of the treatment tool wires 7a and 7b arranged inside the curve formed by the bending, as well as the path lengths of the treatment tool wires 7a and 7b arranged outside the curve formed by the bending, are subjected to change because the lumens 8a and 8b are contracted inside the bending of the guide sheath 8 and are extended outside the bending of the guide sheath 8. In this embodiment, the treatment tool wires 7a and 7b that are arranged outside the curve formed by the bending of the operating bending section 6 are arranged inside the curve formed by the bending of the distal bending section 5, and the treatment tool wires 7a and 7b that are arranged inside the curve formed by the bending of the operating bending section 6 are arranged outside the curve formed by the bending of the distal bending section 5. Thus, the patterns of path-length changes between the treatment tool wires 7a and 7b at the operating bending section 6 and the treatment tool wires 7a and 7b at the distal bending section 5 are opposite. Because the pattern of path-length changes of the treatment tool wires 7a and 7b at the operating bending section 6 is opposite to that of the treatment tool wires 7a and 7b at the distal bending section 5, the changes in the path length of each of the treatment tool wires 7a and 7b are offset overall.

In other words, even when the distal bending section 5 is bent, the overall wire path lengths do not differ between the treatment tool wires 7a and 7b that are arranged inside the curve formed by the bending and the treatment tool wires 7a and 7b that are arranged outside the curve formed by the bending. Therefore, it is possible to prevent a problem that the joint 31 and the grasping section 32 of the treatment section 3 are inadvertently activated when the distal bending section 5 is bent, thereby affording an advantage in that the treatment section 3 can be stably manipulated.

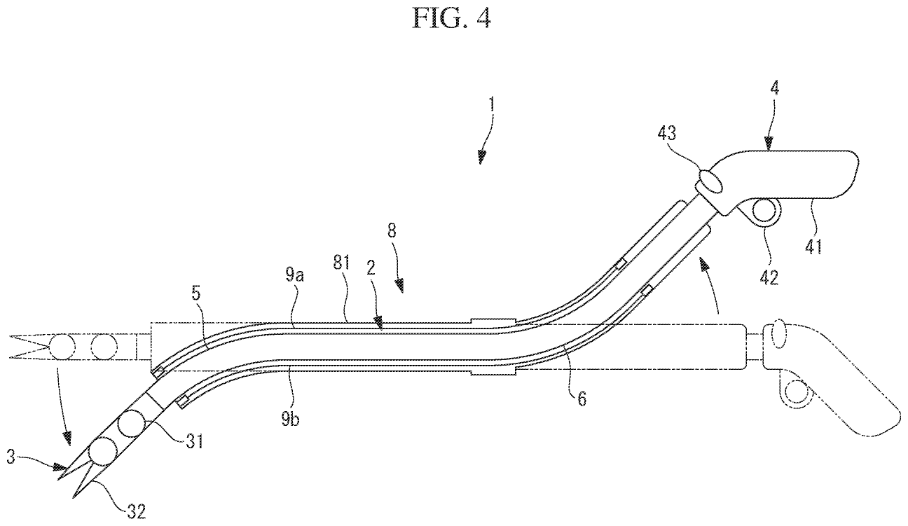

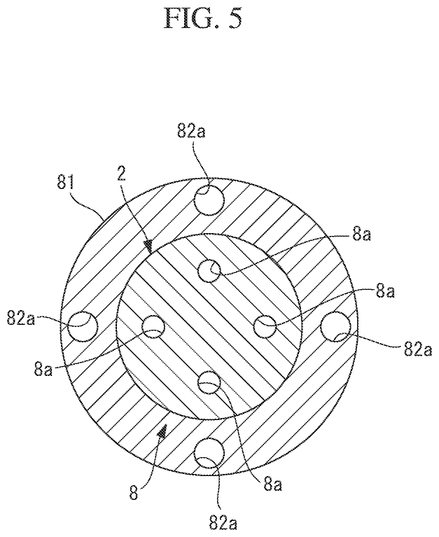

In this embodiment, the members from the handle 41 to the distal bending section 5 are formed of the guide sheath 8, which includes the lumens 8a and 8b for accommodating the treatment tool wires 7a and 7b and the bending wires 9a and 9b. Instead of this, the guide sheath 8 may include the lumens 8a that allow the treatment tool wires 7a and 7b to pass therethrough, and a tubular bending sheath 81 covering the guide sheath 8 may include lumens 82a for accommodating the bending wires 9a and 9b, as shown in FIGS. 4 and 5.

More specifically, the tubular bending sheath 81 is arranged over the handle 41, the operating bending section 6, the shaft 2, and the distal bending section 5; the bending wires 9a and 9b are accommodated along the lumens 82a extending in the longitudinal direction; and both ends of each of the bending wires 9a and 9b are fixed to the distal end and the proximal end, respectively, of the bending sheath 81. On the other hand, the lumens 8a that allow the treatment tool wires 7a and 7b to pass therethrough are provided in the guide sheath 8.

Also with the above-described configuration, even when the distal bending section 5 is bent in the same manner as in the above-described embodiment, the overall wire path lengths do not differ between the treatment tool wires 7a and 7b that are arranged inside the curve formed by the bending and the treatment tool wires 7a and 7b that are arranged outside the curve formed by the bending. Therefore, it is possible to prevent a problem that the joint 31 and the grasping section 32 of the treatment section 3 are inadvertently activated when the distal bending section 5 is bent, thereby affording an advantage in that the treatment section 3 can be stably manipulated.

In addition, there is another advantage in that even when the treatment section 3 is rotated in the bending sheath 81 in a state in which the distal bending section 5 is bent, the path lengths do not differ.

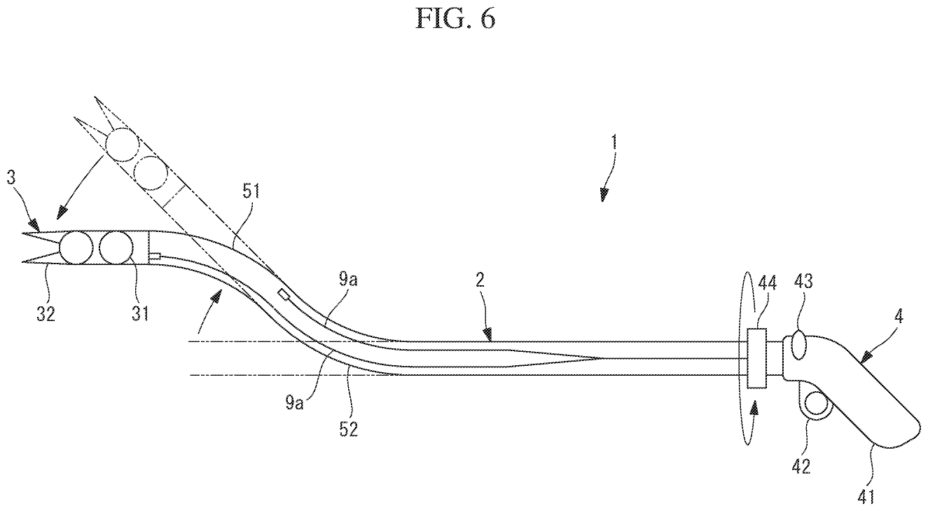

In addition, in this embodiment, the distal bending section 5 is provided at the distal end of the rigid shaft 2, the operating bending section 6 is provided at the proximal end of the rigid shaft 2, and the distal bending section 5 is bent by swiveling the operating section 4. Instead of this, two bending sections (a first bending section and a second bending section) 51 and 52 may be provided, so as to be spaced apart from each other in the length direction, in the guide sheath 8 that is arranged at a more distal position than the shaft 2, as shown in FIG. 6. With this configuration, the two bending sections 51 and 52 can be bent simultaneously by means of a dial 44 provided on the operating section 4.

In this case, the bending wires 9a and 9b, which are connected to the two bending sections 51 and 52, are connected to the dial 44, and when the dial 44 is rotated about the longitudinal axis of the shaft 2, the bending wires 9a and 9b are pulled or relaxed. For example, in order to simultaneously bend the two bending sections 51 and 52 in opposite directions in the same bending plane by operating the dial 44, the two bending wires 9a to be pulled are connected to the inner sides of the curves formed by the bending, and the two bending wires 9b to be relaxed are connected to the outer sides of the curves formed by the bending. FIG. 6 shows only the bending wires 9a that are pulled.

When the phase shift of arrangement of the treatment tool wires 7a and 7b is made to coincide with that of the bending wires 9a and 9b at the two bending sections 51 and 52, the path lengths do not differ between the treatment tool wires 7a and 7b arranged inside and outside the curves formed by the bending. This affords an advantage in that the treatment section 3 can be stably manipulated. The same advantage can also be afforded in the case where the bending sheath 81 as shown in FIG. 4 is used.

In addition, although this embodiment has been described by way of an example of the guide sheath 8 having the plurality of lumens 8a and 8b, instead of this, the treatment tool wires 7a and 7b may be accommodated in individual guide sheaths for allowing the treatment tool wires 7a and 7b individually to pass therethrough. In addition, in this case, the guide sheaths at one of the bending sections may be formed of a coil sheath 53, and the guide sheaths at the other of the bending sections may be formed of a soft tube 54.

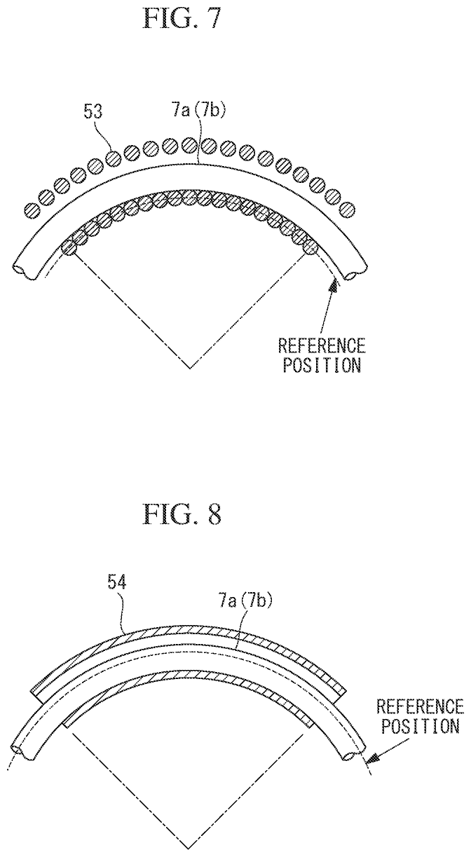

When the coil sheath 53 is to be bent, the coils inside the curve formed by the bending are maintained closely packed, whereas the coils outside the curve formed by the bending are spaced widely, as shown in FIG. 7. Therefore, the path length of each of the treatment tool wires 7a and 7b passing through the coil sheath 53 increases with respect to the line (broken line) connecting the coils closely packed inside the curve.

On the other hand, when the soft tube 54 is to be bent, the inner side of the curve formed by the bending contracts, and the outer side of the curve extends, as shown in FIG. 8, causing each of the treatment tool wires 7a and 7b to be decentered towards the inner side of the curve formed by the bending. Therefore, the path length of each of the treatment tool wires 7a and 7b decreases with respect to the center line (broken line) of the tube 54. Therefore, the path-length changes of each of the treatment tool wires 7a and 7b can be offset.

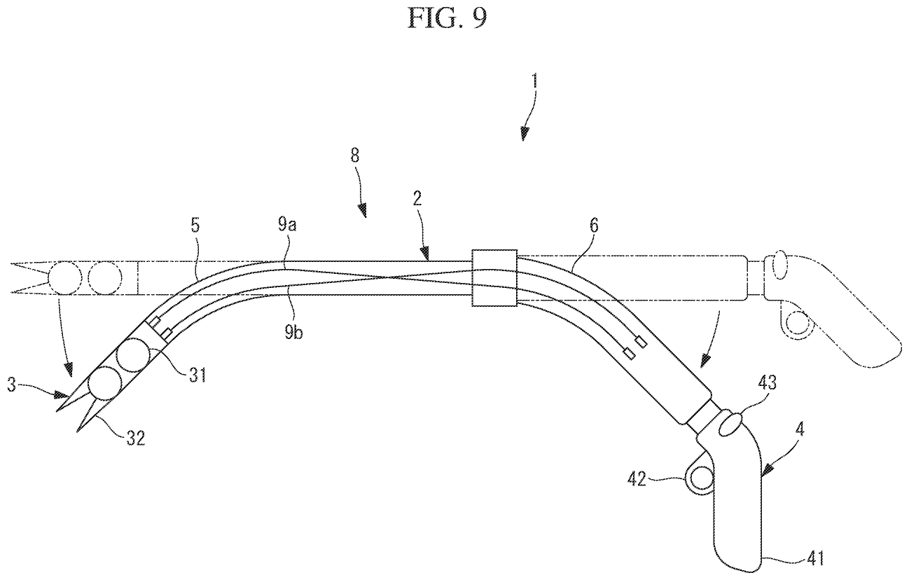

In addition, although this embodiment has been described by way of an example where the two bending sections 5 and 6 are bent in opposite directions in the same bending plane, instead of this, the two bending sections 5 and 6 may be bent in the same direction in the same bending plane, as shown in FIG. 9, by twisting the lumens 8a and 8b, which allow the bending wires 9a and 9b and the treatment tool wires 7a and 7b to pass therethrough, by 180° at the shaft 2. By doing so, when the operator operates the operating section 4 downward, the treatment section 3 is also bent downward. This affords an advantage in allowing intuitive operation.

Note that, in the case where the distal bending section 5 and the operating bending section 6 are bent in different bending planes from each other, it is advisable that the twist angle of the lumens 8a and 8b at the shaft 2 be set according to the angle between the bending planes. This affords an advantage in that the direction in which the operating section 4 is operated by the operator can be set to any easy-to-operate direction.

In addition, the lumens 8a and 8b for the treatment tool wires 7a and 7b and the bending wires 9a and 9b may be further twisted by a multiple of 360°, as long as the state in which the phases of both ends of each of the treatment tool wires 7a and 7b and the bending wires 9a and 9b are set at the twist angle according to the angle between the bending planes of the two bending sections 5 and 6 is maintained. By doing so, initial path-length changes can be reduced.

In addition, although this embodiment has been described by way of an example where the distal bending section 5 and the operating bending section 6 have the same curvature angle and also have the same radius (bending radius) of the lumens 8a for the treatment tool wires 7a and 7b as measured from the center of the guide sheath 8, instead of this, the distal bending section 5 and the operating bending section 6 may have different radii and curvature angles, as long as conditional expression (1) below is satisfied.

r1θ1=r2θ2 (1)

Here, r1 is the radius of a lumen 8a as measured from the center of the guide sheath 8 at the distal bending section 5, r2 is the radius of a lumen 8a as measured from the center of the guide sheath 8 at the operating bending section 6, 01 is the curvature angle at the distal bending section 5, and 02 is the curvature angle at the operating bending section 6.

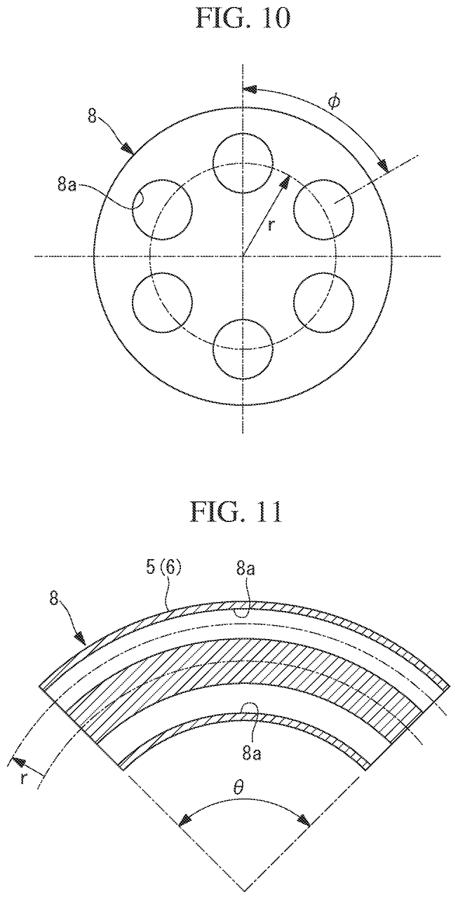

As shown in FIGS. 10 and 11, a path length change dL due to bending is expressed by the following expression.

dL=rθ cos φ

Here, the phase of lumens 8a is φ, the radius of a lumen 8a as measured from the center of the guide sheath 8 is r, and the curvature angle is 0.

If the phase of the lumens 8a is the same at the two bending sections 5 and 6, conditional expression (1) holds in order to make the path length change at the bending section 5 equivalent to that at the bending section 6.

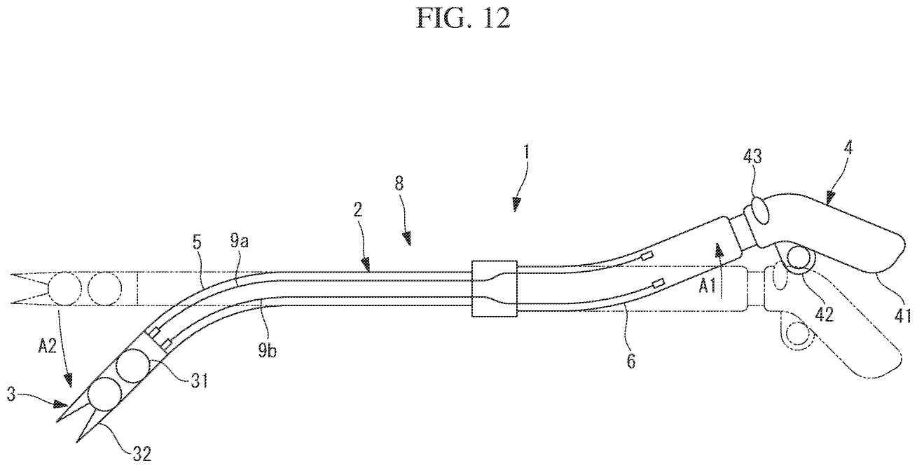

Therefore, as shown in FIG. 12, the curvature angle at the operating bending section 6 can be made smaller by making the radius r2 of a lumen 8a, as measured from the center of the guide sheath 8 at the operating bending section 6, larger than the radius r1 at the distal bending section 5. By doing so, a large swiveling angle A2 of the treatment section 3 at the distal bending section 5 can be ensured with even a smaller operation angle A1.

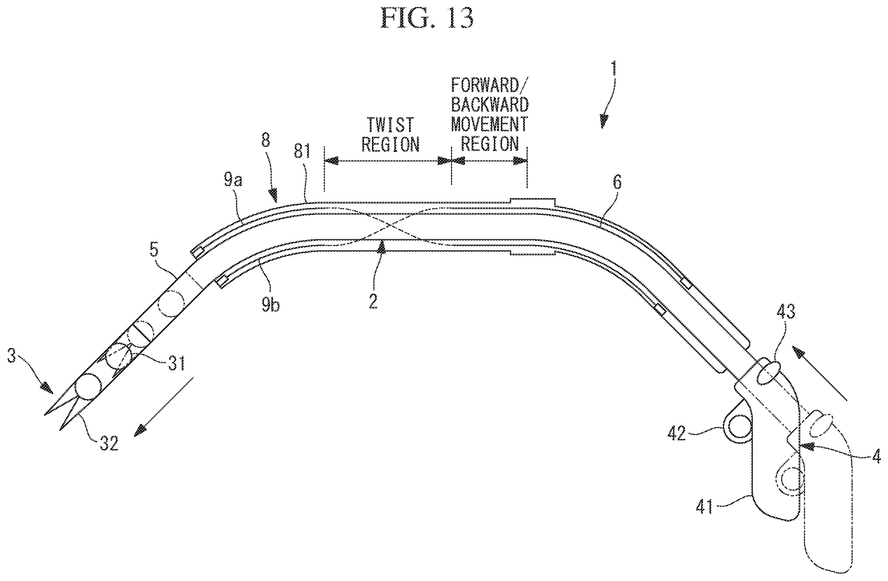

In addition, as shown in FIG. 13, the guide sheath 8 may be arranged so that the guide sheath 8 can be moved forward/backward in the longitudinal direction relative to the bending sheath 81. On this configuration, in the case where the lumens 8a and 8b for the treatment tool wires 7a and 7b and the bending wires 9a and 9b are twisted, it is necessary to arrange the guide sheath 8 at a position at which, even when the guide sheath 8 is moved forward/backward by the maximum possible amount, the twist region does not overlap either of the bending sections 5 and 6. By doing so, the path length changes can be offset regardless of the forward/backward movement position of the treatment section 3.

The above-described embodiments are derived from the following aspects of the present invention.

One aspect of the present invention is a medical treatment tool including: a treatment section that can treat an affected area; an operating section for operating the treatment section; a guide sheath arranged between the treatment section and the operating section; a first bending section and a second bending section for bending the guide sheath, the first bending section and the second bending section being arranged between the operating section and the treatment section in a manner spaced apart from each other in a longitudinal direction; and at least two force transmission members that pass through the first bending section and the second bending section of the guide sheath and that transmit, to the treatment section, a force applied to the operating section, wherein the first bending section and the second bending section are bent in directions in which changes in path lengths of the force transmission members in the guide sheath caused by bending of the first bending section are offset by changes in the path lengths of the force transmission members in the guide sheath caused by bending of the second bending section.

According to this aspect, by operating the operating section arranged outside the body while the treatment section is arranged inside the body, the force applied to the operating section is transmitted to the treatment section via the at least two force transmission members that are arranged so as to pass through the first bending section and the second bending section of the guide sheath arranged between the treatment section and the operating section. By doing so, the affected area can be treated by operating the treatment section.

In this case, when the first bending section and the second bending section are bent, the first bending section and the second bending section are bent in directions in which the path-length changes of the force transmission members in the guide sheath caused by the bending of the first bending section are offset by the path-length changes of the force transmission members in the guide sheath caused by the bending of the second bending section. More specifically, in the case where one of the force transmission members is arranged inside the curve formed by the bending at the first bending section and the other of the force transmission members is arranged outside the curve formed by the bending at the first bending section, the second bending section is bent in a direction in which the one of the force transmission members is arranged outside the curve formed by the bending at the second bending section and in which the other of the force transmission members is arranged inside the curve formed by the bending at the second bending section. This allows the path-length changes of the guide sheath to be offset as a whole, preventing the treatment section from being manipulated in an unexpected direction.

According to this aspect, the paths of the force transmission members are not twisted or are twisted at a long pitch by exploiting the relationship between the first bending section and the second bending section, instead of being twisted at a short pitch in the narrow area of the first bending section. This makes it possible to suppress a path-length change caused by bending while suppressing an increase in friction.

In the above-described aspect, the first bending section may be bent by bending the second bending section by operating the operating section.

With this configuration, when the operating section is swiveled, the second bending section is bent, and path-length changes caused by bending of the first bending section can be offset by exploiting the bending of the second bending section.

In addition, in the above-described aspect, bending lengths and curvatures of the guide sheath at the first bending section and the second bending section may be the same.

With this configuration, path-length changes of the force transmission members can be offset by bending the first bending section by the same angle as the curvature angle of the second bending section.

In addition, in the above-described aspect, products of bending radii and curvature angles of paths of the force transmission members in the guide sheath at the first bending section and the second bending section are preferably the same.

With this configuration, it is possible to make the curvature angle of the first bending section different from the curvature angle of the second bending section by making the bending radii of the paths of the force transmission members different between the first bending section and the second bending section.

In addition, in the above-described aspect, the first bending section and the second bending section may be bent in the same direction or opposite directions in the same plane.

With this configuration, twisting the paths of the force transmission members is avoided or the paths of the force transmission members is merely twisted by 180°, whereby it is possible to suppress a path-length change caused by bending while ensuring a long twist pitch and suppressing an increase in friction.

In addition, in the above-described aspect, the guide sheath may be a multi-lumen tube with a plurality of lumens that allow the force transmission members to pass therethrough, the lumens of the guide sheath may extend in a straight line between the first bending section and the second bending section, and the first bending section and the second bending section may be bent in opposite directions in the same plane.

With this configuration, twisting the paths of the force transmission members is avoided, whereby it is possible to suppress a path-length change caused by bending while suppressing an increase in friction.

In addition, in the above-described aspect, the guide sheath may be a multi-lumen tube with a plurality of lumens that allow the force transmission members to pass therethrough, and, with respect to a position at which the first bending section and the second bending section are bent in opposite directions in the same plane, the lumens of the guide sheath may be twisted between the first bending section and the second bending section in the same direction by the same angle that the swiveling plane of the operating section is twisted relative to the same plane.

With this configuration, even when the bending direction of the first bending section and the bending direction of the second bending section are set arbitrarily, it is possible to suppress a path-length change caused by the bending while suppressing an increase in friction. By doing so, the operating direction of the operating section can be set in an arbitrary easy-to-operate direction.

In addition, in the above-described aspect, the guide sheath may be a multi-lumen tube with a plurality of lumens that allow the force transmission members to pass therethrough, each of the lumens may be twisted about the longitudinal axis of the guide sheath, and the phases of twist of the lumens relative to the bending direction of the first bending section may be opposite to the phases of twist of the lumens relative to the bending direction of the second bending section.

With this configuration, even when the bending direction of the first bending section and the bending direction of the second bending section are set arbitrarily, it is possible to suppress a path-length change caused by the bending while suppressing an increase in friction. By doing so, the operating direction of the operating section can be set in an arbitrary easy-to-operate direction.

In addition, the above-described aspect may further include: a bending sheath that allows the guide sheath to pass therethrough, wherein the bending sheath may be bent by bending the second bending section by operating the operating section, and the first bending section may be bent according to bending of the bending sheath.

With this configuration, also in the case where the guide sheath passing through the bending sheath is indirectly bent by bending the bending sheath, it is possible to offset path-length changes of the force transmission members by means of the first bending section and the second bending section.

In addition, in the above-described aspect, bending lengths and curvatures of the guide sheath at the first bending section and the second bending section may be the same.

In addition, in the above-described aspect, the products of bending radii and curvature angles of the paths of the force transmission members in the guide sheath at the first bending section and the second bending section are preferably the same.

In addition, in the above-described aspect, the first bending section and the second bending section may be bent in the same direction or opposite directions in the same plane.

In addition, in the above-described aspect, the guide sheath may be a multi-lumen tube with a plurality of lumens that allow the force transmission members to pass therethrough, the lumens of the guide sheath may extend in a straight line between the first bending section and the second bending section, and the first bending section and the second bending section may be bent in opposite directions in the same plane.

In addition, in the above-described aspect, the guide sheath may be a multi-lumen tube with a plurality of lumens that allow the force transmission members to pass therethrough, and, with respect to a position at which the first bending section and the second bending section are bent in opposite directions in the same plane, the lumens of the guide sheath may be twisted between the first bending section and the second bending section in the same direction by the same angle that the swiveling plane of the operating section is twisted relative to the same plane.

In addition, in the above-described aspect, the guide sheath may be a multi-lumen tube with a plurality of lumens that allow the force transmission members to pass therethrough, each of the lumens may be twisted about the longitudinal axis of the guide sheath, and the phases of twist of the lumens relative to the bending direction of the first bending section may be opposite to the phases of twist of the lumens relative to the bending direction of the second bending section.

In addition, in the above-described aspect, the guide sheath may be a multi-lumen tube with a plurality of lumens that allow the force transmission members to pass therethrough, the treatment section and the guide sheath may be arranged so as to be movable back and forth in the longitudinal direction relative to the bending sheath, and twist of the lumens of the guide sheath may be arranged to be kept between the first bending section and the second bending section.

With this configuration, it is possible to move the guide sheath back and forth relative to the bending sheath, making it possible to suppress a path-length change caused by bending while suppressing an increase in friction even when the first bending section and the second bending section are bent at any forward/backward movement position.

### REFERENCE SIGNS LIST

1 Medical treatment tool
2 shaft
3 Treatment section
4 Operating section
5 Distal bending section (first bending section)
51, 52 Bending section (first bending section, second bending
section)
6 Operating bending section (second bending section)
7a, 7b Treatment tool wire (force transmission member)
8 Guide sheath (multi-lumen tube)
8a, 8b Lumen
81 Bending sheath
9a, 9b bending wire
r1, r2 Radius (bending radius)
θl, θ2 Curvature angle

1. A medical treatment tool comprising:
a forceps that treats an affected area;
an operating section comprising a handle and a dial, and configured to operate the forceps;
a guide sheath arranged between the forceps and the operating section;
a first bending section and a second bending section arranged between the operating section and the forceps in a manner spaced apart from each other in a longitudinal direction, the first bending section and the second bending section bending the guide sheath;
at least two treatment tool wires that pass through the first bending section and the second bending section of the guide sheath and that transmit, to the forceps, a force applied to the operating section; and
at least two bending wires that pass through the first bending section and the second bending section of the guide sheath and that are fixed to the operating section and the forceps, the bending wires causing the guide sheath to be bent by operating the operating section,
wherein the guide sheath comprises a multi-lumen tube with a plurality of lumens that allow the treatment tool wires to pass therethrough,
with respect to a position of lumens at which the first bending section and the second bending section are bent in opposite directions on the same plane, the lumens of the guide sheath are twisted between the first bending section and the second bending section in the same direction by the same angle that operating direction of the operating section is twisted relative to the opposite direction mentioned above, and
the first bending section and the second bending section are bent in directions in which changes in path lengths of the treatment tool wires in the guide sheath caused by bending of the first bending section are offset by changes in the path lengths of the treatment tool wires in the guide sheath caused by bending of the second bending section.

2. The medical treatment tool according to claim 1, wherein the first bending section is bent by bending the second bending section by operating the operating section.

3. The medical treatment tool according to claim 2, wherein bending lengths and curvatures of the guide sheath at the first bending section and the second bending section are the same.

4. The medical treatment tool according to claim 2, wherein products of bending radii and curvature angles of paths of the treatment tool wires in the guide sheath at the first bending section and the second bending section are the same.

5. The medical treatment tool according to claim 1, further comprising:
a bending sheath that allows the guide sheath to pass therethrough,
wherein the bending sheath is bent by bending the second bending section by operating the operating section, and the first bending section is bent according to bending of the bending sheath.

6. The medical treatment tool according to claim 5, wherein bending lengths and curvatures of the guide sheath at the first bending section and the second bending section are the same.

7. The medical treatment tool according to claim 5, wherein products of bending radii and curvature angles of paths of the treatment tool wires in the guide sheath at the first bending section and the second bending section are the same.

8. A medical treatment tool comprising:
a forceps that treats an affected area;
an operating section comprising a handle and a dial, and configured to operate the forceps;
a guide sheath arranged between the forceps and the operating section;
a first bending section and a second bending section arranged between the operating section and the forceps in a manner spaced apart from each other in a longitudinal direction, the first bending section and the second bending section bending the guide sheath;
at least two treatment tool wires that pass through the first bending section and the second bending section of the guide sheath and that transmit, to the forceps, a force applied to the operating section;
a bending sheath that allows the guide sheath to pass therethrough; and
at least two bending wires that pass through the bending sheath and that are fixed to a distal end and a proximal end of bending sheath, the bending wires causing the guide sheath to be bent by operating the operating section,
wherein the bending sheath is bent by bending the second bending section by operating the operating section, and the first bending section is bent according to bending of the bending sheath,
the guide sheath comprises a multi-lumen tube with a plurality of lumens that allow the treatment tool wires to pass therethrough,
the forceps and the guide sheath are arranged so as to be movable back and forth in the longitudinal direction relative to the bending sheath,
twist of the lumens of the guide sheath is arranged to be kept between the first bending section and the second bending section, and
the first bending section and the second bending section are bent in directions in which changes in path lengths of the treatment tool wires in the guide sheath caused by bending of the first bending section are offset by changes in the path lengths of the treatment tool wires in the guide sheath caused by bending of the second bending section.

9. The medical treatment tool according to claim 8, wherein bending lengths and curvatures of the guide sheath at the first bending section and the second bending section are the same.

10. The medical treatment tool according to claim 8, wherein products of bending radii and curvature angles of paths of the treatment tool wires in the guide sheath at the first bending section and the second bending section are the same.
